(19) United States
(12) Patent Application Publication
(10) Pub. No.: US 2010/0311114 A1
(43) Pub. Date: Dec. 9, 2010

(54) PREPARATION OF SAMPLES FOR PROTEOME ANALYSIS

(75) Inventors: Sven Eyckerman, De Pinte (BE); Koen Kas, Schilde (BE)

Correspondence Address: KNOBBE MARTENS OLSON & BEAR LLP, 2040 MAIN STREET, FOURTEENTH FLOOR, IRVINE, CA, 92614, US

(73) Assignee: Pronota N.V., Zwijnaarde (BE)

(21) Appl. No.: 12/738,266

(22) PCT Filed: Oct. 17, 2008
(86) PCT No.: PCT/EP2008/064031

§ 371 Date: Apr. 15, 2010

(30) Foreign Application Priority Data
Oct. 19, 2007 (EP) 07118859.3
Jan. 31, 2008 (EP) 08150909.3

(51) Int. Cl. C12P 21/00; C12N 9/48

(52) U.S. Cl. 435/68.1; 435/212

(57) ABSTRACT

The invention mainly concerns methods and systems for the preparation of biological samples for proteome analysis, N-terminal or C-terminal peptides of proteins are enriched using exopeptidase.

### FIELD OF THE INVENTION

The invention relates to the field of proteomics. Methods and systems for the preparation of biological samples for proteome analysis, and for identification and quantification of proteins and peptides from such samples are provided.

### BACKGROUND OF THE INVENTION

The proteome is usually described as the entire complement of proteins found in a biological system, such as, e.g., a cell, tissue, organ or organism. Proteomics is concerned with the study of the proteome expressed at particular times and/or under internal or external conditions of interest. Proteomics approaches frequently aim at global analysis of the proteome, and require that large numbers of proteins, e.g., hundreds or thousands, can be routinely resolved and identified from a single sample.

Among the promises of proteomics is its ability to recognise new biomarkers, i.e., biological indicators that signal a changed physiological state, such as due to a disease or a therapeutic intervention. Biomarker discovery usually involves comparing proteomes expressed in distinct physiological states, and identifying proteins whose occurrence or expression levels consistently differ between said physiological states.

Methods allowing for proteome analysis without the need to purify each protein to homogeneity have been developed. Typically, such methods fragment the proteins of a sample into peptides using agents with known specificity of cleavage (e.g., endoproteinases), fractionate the constituent peptides by chromatography, and determine the mass and, optionally, sequence of peptides present in the diverse fractions by mass spectrometry. The obtained mass and sequence information is used to search sequence databases in order to identify proteins from which the respective peptides originated.

However, proteolysis of complex biological samples can produce thousands of peptides, which may overwhelm the resolution capacity of current chromatographic and mass spectrometric systems, causing incomplete coverage and impaired identification of the constituent peptides.

One manner to enable proteome analysis of biological samples is to reduce the complexity of protein peptide mixtures generated by fragmentation of such samples, before subjecting said peptide mixtures to downstream resolving and identification steps, such as chromatographic separation and/or MS. Ideally, reducing the complexity of protein peptide mixtures will decrease the average number of distinct peptides present per individual protein of the sample, yet will maximise the fraction of proteins of the sample actually represented in the peptide mixture.

WO 02/077016 disclosed a proteomics approach wherein the complexity of a peptide mixture was reduced by: (i) resolving the mixture into fractions, (ii) chemically or enzymatically labelling each of said fractions, and (iii) isolating the desired subset of peptides (labelled or unlabelled) from each fraction using a resolution step substantially similar to that of step (i). Although valuable, the method involves multiple handling steps, which might introduce errors and increase labour-intensiveness. In addition, the sensitivity and/or selectivity of this method, as well as of other methods that rely on labelling of either the desired or the remaining peptides, depends on completeness of the respective labelling reactions.

Consequently, there exists a need for further and improved methods that provide effective, robust and relatively simple (e.g., including a minimum of steps and optimally applied on whole peptide digests) manners of reducing the complexity of peptide digests to facilitate comprehensive proteome analysis of biological samples.

### SUMMARY OF THE INVENTION

The invention contemplates innovative methods to enrich peptide mixtures obtained from biological samples for peptides that comprise the N-terminal ends of proteins present in said samples (i.e., N-terminal peptides), or for peptides that comprise the C-terminal ends of proteins present in said samples (i.e., C-terminal peptides).

Given that each protein present in a sample includes an N-terminus and a C-terminus, enrichment for N-terminal peptides or for C-terminal peptides achieves excellent representation of the majority of proteins of said sample and, at the same time, considerable reduction of the complexity of peptide mixtures to be analysed. Also, the present methods can be applied on whole peptide digests, which considerably simplifies the preparation of samples for protein profiling and proteome analysis.

In addition, where a given protein is processed in a biological system by an endogenous proteolytic event into one or more protein fragments, the present methods can also enrich the N-terminal or C-terminal peptides derived from said protein fragments and thus representing the respective endogenous cleavage sites. Hence, the present methods may be particularly useful for analysing proteolytic events occurring in biological systems under normal or physiologically altered conditions. It shall be appreciated that variations in endogenous proteolytic event(s)—that would lead to altered occurrence and/or levels of N-terminal or C-terminal peptide(s) corresponding to the respective cleavage site(s)—may also qualify as potential biomarkers.

Accordingly, in an aspect the invention provides a method for isolating N-terminal peptides from a protein or mixture of proteins, comprising:
(a) protecting the N-terminal amino acid in the protein or in proteins of the protein mixture,
(b) fragmenting the protein or the protein mixture from (a) to obtain a protein peptide mixture, and
(c) reacting the protein peptide mixture from (b) with an aminopeptidase,
   whereby said N-terminal peptides are isolated.

This aspect takes advantage of the situation that fragmentation of a protein in which the N-terminal amino acid has been suitably protected prior to said fragmentation will generate an N-terminal peptide containing a protected N-terminal amino acid, and a C-terminal peptide and optionally one or more internal peptides containing an unprotected amino acid at their respective, newly generated N-termini. Consequently, reacting the protein peptide mixture obtained by fragmentation of said protein with an aminopeptidase leads to hydrolysis (degradation) of the unprotected C-terminal and internal peptides progressively from their respective N-termini into their constituent amino acids. The protected N-terminal peptides of the protein are not degraded by said aminopeptidase, and thereby become enriched or isolated and can be used for downstream analysis.

In another aspect the invention provides a method for isolating C-terminal peptides from a protein or mixture of proteins, comprising:
(a) protecting the C-terminal amino acid in the protein or in proteins of the protein mixture,
(b) fragmenting the protein or the protein mixture from (a) to obtain a protein peptide mixture, and
(c) reacting the protein peptide mixture from (b) with a carboxypeptidase,
   whereby said C-terminal peptides are isolated.

This aspect takes advantage of the situation that fragmentation of a protein in which the C-terminal amino acid has been suitably protected prior to said fragmentation will generate a C-terminal peptide containing a protected C-terminal amino acid, and an N-terminal peptide and optionally one or more internal peptides containing an unprotected amino acid at their respective, newly generated C-termini. Hence, reacting the protein peptide mixture obtained by fragmentation of said protein with a carboxypeptidase leads to hydrolysis (degradation) of the unprotected N-terminal and internal peptides progressively from their respective C-termini into their constituent amino acids. The protected C-terminal peptides of the protein are not degraded by said carboxypeptidase, and thereby become enriched or isolated and can be used for downstream analysis.

Advantageously, the highly efficient and processive action of aminopeptidases and carboxypeptidases can ensure robust, reliable and substantially complete removal of the unprotected peptides, and thereby achieve high degree of enrichment or isolation of, respectively, the protected N-terminal or C-terminal peptides of the starting proteins. Moreover, enzymatic hydrolysis used in the present invention is fairly easy to perform and avoids the need for chemical modifications, which may be rather susceptible to reaction conditions. Also, whereas previous methods relying on separation of peptides labelled with a given moiety from peptides not so-labelled were dependent on the specificity of means of said separation, the present methods degrade the unwanted peptides to their constituent amino acids which substantially do not interfere with downstream analysis of the desired N-terminal or C-terminal peptides. Moreover, if required the amino acids resulting from hydrolysis of the unwanted peptides may be readily separated and removed from the desired N-terminal or C-terminal peptides by common techniques, such as for example RP-chromatography or size exclusion chromatography, on the basis of their different properties, such as, e.g., their considerably smaller size or molecular weight in comparison with peptides).

Hence, the present peptide isolation methods provide robust and straightforward means to isolate or enrich N-terminal or C-terminal peptides from protein peptide mixtures, such as from complex protein peptide mixtures representative of biological samples.

It shall be appreciated that the above methods may be used alone, i.e., wherein the enrichment of N-terminal peptides or C-terminal peptides from the starting proteins is achieved solely by the respective methods. So-isolated N-terminal or C-terminal peptides can then be provided to downstream analysis.

Otherwise, the above methods may be used in conjunction with one or more other peptide sorting methods that enrich or isolate N-terminal or C-terminal peptides; in particular, with methods where the desired N-terminal peptides or C-terminal peptides, but not the remaining unwanted peptides, are already suitably blocked such as to prevent their degradation by aminopeptidases or carboxypeptidases, respectively. The use of the methods of the present invention in conjunction with other peptide sorting strategies can additively or synergically improve the sensitivity and/or specificity of isolating the desired N-terminal or C-terminal peptides.

The above methods have the potential to also recover N-terminal or C-terminal peptides from proteins in which the N-terminal amino acid or the C-terminal amino acid, respectively, is blocked in vivo. This is advantageous vis-à-vis many labelling-based peptide sorting methods, in which such in vivo blocked N-terminal or C-terminal peptides frequently cannot incorporate the label and may thus be lost.

In related aspects, the present methods may be tailored to isolate N-terminal peptides or C-terminal peptides from proteins which are suitably altered in vivo. For example, a considerable portion or even the majority of intracellular proteins in mammalian cells may be in vivo acetylated on their N-terminal α-NH₂ group. In another example, proteins of prokaryotes are translated with a formylated methionine as an initiator for translation, and although the formyl group is typically removed during the translation, it can still be found in some proteins. The deformylase enzyme catalysing the formyl group removal is targeted by next generation antibiotics, underlying the value of tools for monitoring protein deformylation. In yet another example, the activity of glutaminyl cyclase (EC 2.3.2.5) results in the formation of pyroglutaminyl peptides, which is cyclised form of the N-terminal glutamine on some peptides. Activity of this enzyme is described in organ tissues like brain, pituitary, adrenal gland and lymphocytes (Busby et al., 1987, J Biol Chem, 262/15, 8532). These pyroglutaminyl peptides do not have a free amino-terminal amine and they may thus be protected form aminopeptidase activity. In a further example, protein introns or inteins derived from protein splicing include a cyclisation of asparagine (Asn) on their C-terminus. Also in an example, cholesterol modification can occur at the C-terminus and can be sometimes transferred to the C-terminus of an intein of a hedgehog protein.

Where such in vivo protein modification can prevent the action of an aminopeptidase or a carboxypeptidase on N-terminal or C-terminal peptides derived from said proteins and comprising said in vivo modification (such as, e.g., N-terminal α-NH₂ acetylation or N-terminal formylation of proteins or pyroglutaminyl formation on N-terminus of peptides, which can prevent the action of aminopeptidase on their respective N-terminal peptides; or C-terminal Asn cyclisation or C-terminal cholesterol addition of proteins which can prevent the action of carboxypeptidase on their respective C-terminal peptides) the present methods can be adapted to isolate N-terminal or C-terminal peptides carrying such in vivo alteration. This can advantageously allow to analyse the subsets of such in vivo modified proteins.

Accordingly, in an aspect the invention provides a method for isolating, from a protein or mixture of proteins, N-terminal peptides in which the N-terminal amino acid has been blocked in vivo, comprising: (i) fragmenting the protein or the protein mixture to obtain a protein peptide mixture, and (ii) reacting the protein peptide mixture from (i) with an aminopeptidase, whereby said N-terminal peptides in which the N-terminal amino acid has been blocked in vivo are isolated.

In another aspect the invention provides a method for isolating, from a protein or mixture of proteins, C-terminal peptides in which the C-terminal amino acid has been blocked in vivo, comprising: (i) fragmenting the protein or the protein mixture to obtain a protein peptide mixture, and (ii) reacting the protein peptide mixture from (i) with a carboxypeptidase, whereby said C-terminal peptides in which the C-terminal amino acid has been blocked in vivo are isolated.

The term “blocked in vivo” denotes any in vivo modification of a protein's N-terminal or C-terminal amino acid, which can prevent the cleaving-off of said N-terminal or C-terminal amino acid from a protein or peptide containing it by the action of aminopeptidase or carboxypeptidase, respectively. The term “in vivo” generally refers to a living biological system such as, e.g., a cell, a tissue, an organ or an organism, whether in native surroundings or isolated there from (e.g., cell culture). Particularly preferred, although non-limiting, types of in vivo alterations include N-terminal α-NH₂ acetylation or N-terminal formylation of proteins, or pyroglutaminyl formation on N-terminus of peptides, which can prevent the action of aminopeptidase on the respective N-terminal peptides; or C-terminal Asn cyclisation or C-terminal cholesterol addition of proteins which can prevent the action of carboxypeptidase on their respective C-terminal peptides.

As already noted, the present methods can enrich N-terminal or C-terminal peptides that correspond to the N-termini or C-termini of respective full-length proteins, and can also recover N-terminal or C-terminal peptides which correspond to—and thereby identify—proteolytic cleavage events within (full-length) proteins. For example, protein processing or degradation in vivo may produce protein fragments displaying novel N-terminal ends and/or C-terminal ends. The above methods can advantageously follow the appearance of such novel N-terminal or C-terminal peptides which can be identified and may be indicative of novel proteolytic processing events, and/or can follow the changes in absolute or relative quantity of known N-terminal or C-terminal peptides, representative of known cleavage events. Accordingly, the present methods may be advantageously employed in the proteomic study of protein processing (“degradomics”).

By means of example and not limitation, a general approach to identify N-terminal or C-terminal peptides corresponding to proteolytic processing sites may encompass isolating N-terminal or C-terminal peptides of proteins as taught herein, and identifying among so-isolated peptides those which correspond to internal portions of known or predicted full-length proteins.

In further aspects, the subset of N-terminal peptides or C-terminal peptides isolated as taught here above can be subjected to downstream proteome analyses to identify one or more constituent peptides and their corresponding proteins. Typically, this may entail acquiring relevant information for the isolated N-terminal peptides or C-terminal peptides—principally peptide mass and preferably also (partial) peptide sequence—which information allows for database searching to identify the peptides and trace them back to their parent proteins. Accordingly, in an aspect, the methods of the invention may further comprise identifying one or more of the isolated N-terminal peptides or C-terminal peptides, whereby said identified N-terminal peptides or C-terminal peptides represent one or more proteins from the mixture of proteins.

However, given that the complexity of the isolated N-terminal peptides or C-terminal peptides may still be considerable, said peptide identification step may preferably be preceded by a further separation (fractionation) of the peptides using a single- or multi-dimensional separation process. This can further improve the reliability of peptide identification. Accordingly, in an aspect, the methods of the invention may further comprise: (i) separating the isolated N-terminal peptides or C-terminal peptides into fractions of peptides via a single- or multi-dimensional separation process; and (ii) identifying one or more N-terminal peptides or C-terminal peptides from one or more of said fractions, whereby said identified N-terminal peptides or C-terminal peptides represent one or more proteins from the mixture of proteins.

The separation process may resolve the peptides on the basis of one or more physical and/or chemical properties. Exemplary physical and/or chemical properties based on which peptides can be resolved include, without limitation, net charge, electrophoretic mobility (EPM), isoelectric point (pI), molecular size and/or ability or tendency to form certain type(s) of molecular interactions, such as, e.g., hydrogen bonding, dispersive interactions, dipole-dipole polar interactions, dipole-induced dipole polar interactions, ionic interactions, hydrophobic interactions, etc.

Such properties may be evaluated using a variety of separation techniques known per se, including inter alia various electrophoretic and chromatographic separation methods. Preferably, the separation process may comprise or consist of chromatography, such as 1D-, 2D-, 3D- or higher-dimensional chromatography, preferably 1D- or 2D-chromatography, more preferably liquid chromatography.

It shall be appreciated that in the present methods the protein peptide mixture may be treated with aminopeptidase or carboxypeptidase, thereby enriching for N-terminal or C-terminal peptides, respectively, and only thereafter subjected to the above described separation (fractionation) step. This simplifies the handling, since the digest with the aminopeptidase or carboxypeptidase can be performed in a single reaction on the whole protein peptide mixture.

Accordingly, in an aspect the invention provides a method for N-terminal peptide and protein identification and optionally quantification from a mixture of proteins comprising: (a) protecting the N-terminal amino acid in proteins of the protein mixture; (b) fragmenting the protein mixture from (a) to obtain a protein peptide mixture; (c) reacting the protein peptide mixture from (b) with an aminopeptidase, thereby isolating N-terminal peptides; (d) separating the isolated N-terminal peptides into fractions of peptides via a single- or multi-dimensional separation process; and (e) identifying and optionally quantifying one or more N-terminal peptides from one or more of said fractions, whereby said identified N-terminal peptides represent one or more proteins from the mixture of proteins.

Also, in an aspect the invention provides a method for C-terminal peptide and protein identification and optionally quantification from a mixture of proteins comprising: (a) protecting the C-terminal amino acid in proteins of the protein mixture; (b) fragmenting the protein mixture from (a) to obtain a protein peptide mixture; (c) reacting the protein peptide mixture from (b) with a carboxypeptidase, thereby isolating C-terminal peptides; (d) separating the isolated C-terminal peptides into fractions of peptides via a single- or multi-dimensional separation process; and (e) identifying and optionally quantifying one or more C-terminal peptides from one or more of said fractions, whereby said identified C-terminal peptides represent one or more proteins from the mixture of proteins.

An exemplary but non-limiting illustration of this sequence of actions is shown in FIG. 1A for aminopeptidase.

Otherwise, it is also contemplated to first separate (fractionate) the protein peptide mixture into fractions of peptides using the above described separation step, and only thereafter treat said fraction(s) with aminopeptidase or carboxypeptidase to isolate N-terminal peptides or C-terminal peptides there from, respectively. Such sequence of actions may, e.g., allow to perform the reaction with amino- or carboxypeptidase on a limited number of fractions of interest, thereby reducing the reaction volumes and need for reagents.

Accordingly, in an aspect the invention provides a method for N-terminal peptide and protein identification and optionally quantification from a mixture of proteins comprising: (x) protecting the N-terminal amino acid in proteins of the protein mixture; (y) fragmenting the protein mixture from (x) to obtain a protein peptide mixture; (z) separating the protein peptide mixture from (y) into fractions of peptides via a single- or multi-dimensional separation process; (u) reacting one or more fractions from (z) with an aminopeptidase, thereby isolating N-terminal peptides; and (w) identifying and optionally quantifying one or more N-terminal peptides from one or more fractions of (u), whereby said identified N-terminal peptides represent one or more proteins from the mixture of proteins.

Also, in an aspect the invention provides a method for C-terminal peptide and protein identification and optionally quantification from a mixture of proteins comprising: (x) protecting the C-terminal amino acid in proteins of the protein mixture; (y) fragmenting the protein mixture from (x) to obtain a protein peptide mixture; (z) separating the protein peptide mixture from (y) into fractions of peptides via a single- or multi-dimensional separation process; (u) reacting one or more fractions from (z) with a carboxypeptidase, thereby isolating C-terminal peptides; and (w) identifying and optionally quantifying one or more C-terminal peptides from one or more fractions of (u), whereby said identified C-terminal peptides represent one or more proteins from the mixture of proteins.

An exemplary but non-limiting illustration of this sequence of actions is shown in FIG. 1B for aminopeptidase.

In a further aspect, the invention provides a kit specifically designed for isolating N-terminal peptides or C-terminal peptides from a protein or mixture of proteins, comprising one or more or all of the following elements:
an agent for effecting protection of the N-terminal amino acid in the protein or in proteins of the protein mixture, and/or an agent for effecting protection of the C-terminal amino acid in the protein or in proteins of the protein mixture, as defined herein;
an agent for effecting fragmentation of the protein or the protein mixture into a protein peptide mixture, as defined herein;
one or more aminopeptidases and/or one or more carboxypeptidases, as defined herein;
a separation means for separating peptides and amino acids, preferably a size exclusion chromatographic means, such as, more preferably, a size exclusion chromatographic column, said size exclusion chromatographic means having a separation cut-off of between about 400 Da and about 1000 Da, more preferably between about 500 Da and about 800 Da, even more preferably of about 600 Da or about 700 Da.

In a further aspect, the invention provides a means or device, such as preferably an automatic processing station, configured to isolate N-terminal peptides or C-terminal peptides from a protein or mixture of proteins using the methods of the invention. It shall be appreciated that the invention is also directed to a peptide sorting device or system that is configured to perform the methods of the invention, in particular to isolate N-terminal peptides or C-terminal peptides from a protein or mixture of proteins as taught herein, followed by single- or multi-dimensional separation of the isolated peptides, and optionally identification of one or more of said peptides. Hence, the invention also relates to a system for identification of peptides comprising: a means or device, such as preferably an automatic processing station, configured to isolate N-terminal peptides or C-terminal peptides from a protein or mixture of proteins using the methods of the invention; and one or more downstream chromatographic columns for separating the N-terminal peptides or C-terminal peptides into a plurality of fractions in a single- or multi-dimensional separation process; and optionally a downstream mass spectrometric analyser. Preferably, the system may be configured to perform any two or more or all above peptide sorting and separation steps “in-line”, i.e., by directly feeding desired analytes from a previous separation element to the subsequent separation element.

The invention also contemplates use of the present methods and systems to identify proteins differentially present between different samples, preferably to identify biomarkers.

The invention also contemplates use of the present methods and systems to identify endogenous proteolytic events and cleavage sites in proteins, for example to identify such differentially present endogenous proteolytic events and cleavage sites in proteins between different samples.

These and further aspects and preferred embodiments of the invention are described in the following sections and in the appended claims.

### BRIEF DESCRIPTION OF FIGURES

FIG. 1 A, B schematically illustrate proteomic analysis involving aminopeptidase-facilitated isolation of N-terminal peptides.

FIG. 2: MALDI profiles from untreated peptide mixtures (A), or from peptide mixtures treated with Aeromonas proteolytica aminopeptidase (B). The 1558 and 1928 masses represent the two acetylated peptides. The 1841 mass corresponds to an acetylated contaminant peptide in one of the spiked peptides (i.e. the 1928 mass minus 1 amino acid).

FIG. 3: MALDI mass spectra from peptide mixes treated overnight at 37° C. with 0.05U dialyzed aminopeptidase M (panel B) compared to the untreated peptide mix (panel A). The arrows marked with the asterisk point to two ICPL-modified peptides. The unmarked arrows show 3 masses from the Pepmix4 peptides. Aminopeptidase treatment results in complete removal of the unprotected peptides.

FIG. 4: MALDI profiles of treated (B) vs untreated peptide mixes (A). Short incubation with aminopeptidase M leads to drastic removal of peptides from the mass plot. The arrows marked with an asterisk point to the acetylated peptides that were added. The remaining arrows point to acetylated N-termini from 3/7 proteins that fall within the detection window. Panels (C) to (E) and (F) to (H) show zoomed in regions from panel (A) en panel (B), respectively. The arrows in panels (C) to (H) point to the mass of the expected acetylated N-terminus from 3 different proteins that fall within the detection window of the analysis.

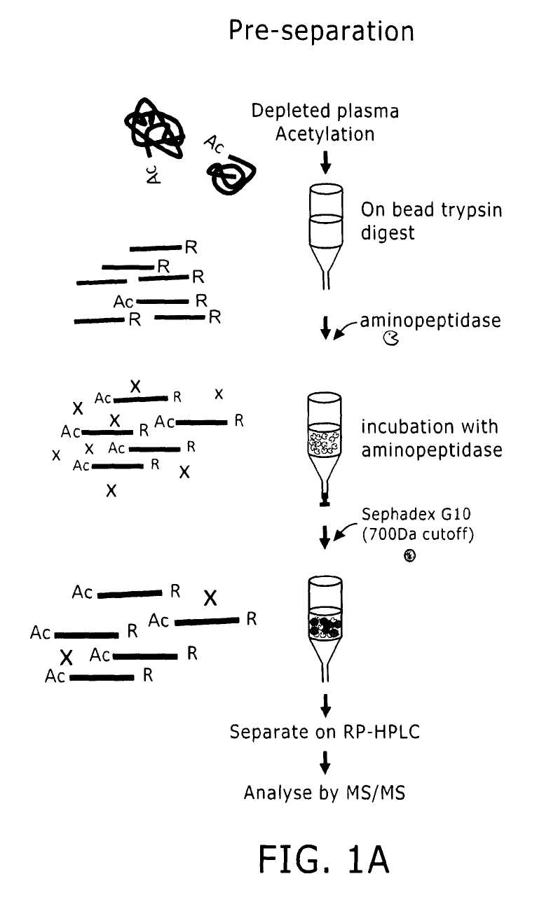

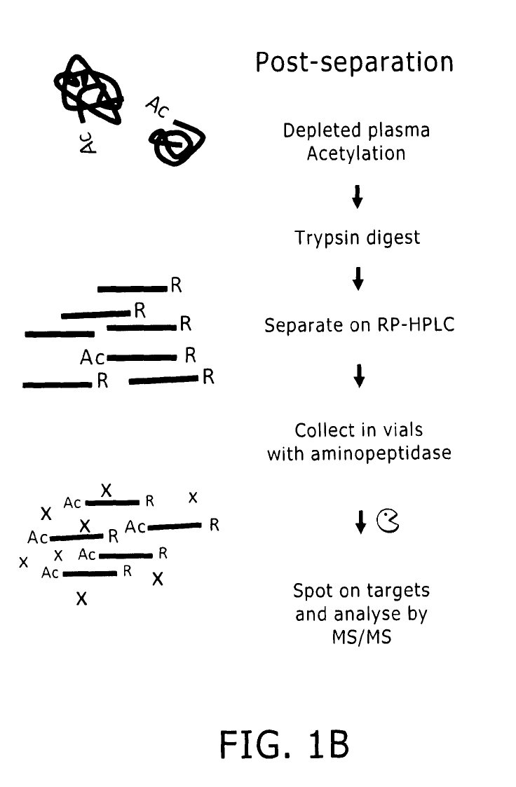

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

When referring to a group of members or entities throughout this specification, “substantially all” means 70% or more, e.g., 75% or more, preferably 80% or more, e.g., 85% or more, more preferably 90% or more, even more preferably 95% or more, and most preferably at least 96%, at least 97%, at least 98%, at least 99% or even 100% of said members or entities.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such connotation is meant to apply throughout this specification, i.e., also in the context of other aspects or embodiments, unless otherwise defined.

### Analysed Samples

The term “protein” as used herein refers to naturally or recombinantly produced macromolecules comprising one or more polypeptide chains, i.e., polymeric chains of amino acid residues linked by peptide bonds. The term thus encompasses monomeric proteins, as well as protein dimers (hetero- as well as homo-dimers) and protein multimers (hetero- as well as homo-multimers). Further, the term also encompasses proteins that carry one or more co- or post-expression modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. In addition, the term includes nascent protein chains as well as partly or wholly folded proteins, misfolded proteins, partly or wholly unfolded or denatured proteins, and may also cover coalesced or aggregated proteins, in particular where the latter are amenable to proteolysis. The term further also includes protein variants or mutants which carry amino acid sequence variations vis-à-vis a corresponding native protein, such as, e.g., amino acid deletions, additions and/or substitutions. The term contemplates both full-length proteins and protein parts, preferably naturally-occurring protein parts that ensue from further processing of said full-length proteins.

The invention may analyse a single protein (e.g., gel-excised protein) and is particularly suitable for analysing mixtures of proteins, including complex protein mixtures. The terms “mixture of proteins” or “protein mixture” generally refer to a mixture of two or more different proteins, e.g., a composition comprising said two or more different proteins.

In preferred embodiments, a mixture of proteins to be analysed herein may include more than about 10, preferably more than about 50, even more preferably more than about 100, yet more preferably more than about 500 different proteins, such as, e.g., more than about 1000 or more than about 5000 different proteins.

An exemplary complex protein mixture may involve, without limitation, all or a fraction of proteins present in a biological sample or part thereof. The terms “biological sample” or “sample” as used herein generally refer to material, in a non-purified or purified form, obtained from a biological source. By means of example and not limitation, samples may be obtained from: viruses, e.g., viruses of prokaryotic or eukaryotic hosts; prokaryotic cells, e.g., bacteria or archeae, e.g., free-living or planktonic prokaryotes or colonies or bio-films comprising prokaryotes; eukaryotic cells or organelles thereof, including eukaryotic cells obtained from in vivo or in situ or cultured in vitro; eukaryotic tissues or organisms, e.g., cell-containing or cell-free samples from eukaryotic tissues or organisms; eukaryotes may comprise protists, e.g., protozoa or algae, fungi, e.g., yeasts or molds, plants and animals, e.g., mammals, humans or non-human mammals. Biological sample may thus encompass, for instance, a cell, tissue, organism, or extracts thereof. A biological sample may be preferably removed from its biological source, e.g., from an animal such as mammal, human or non-human mammal, by suitable methods, such as, without limitation, collection or drawing of urine, saliva, sputum, semen, milk, mucus, sweat, faeces, etc., drawing of blood, cerebrospinal fluid, interstitial fluid, optic fluid (vitreous) or synovial fluid, or by tissue biopsy, resection, etc. A biological sample may be further subdivided to isolate or enrich for parts thereof to be used for obtaining proteins for analysing in the invention. By means of example and not limitation, diverse tissue types may be separated from each other; specific cell types or cell phenotypes may be isolated from a sample, e.g., using FACS sorting, antibody panning, laser-capture dissection, etc.; cells may be separated from interstitial fluid, e.g., blood cells may be separated from blood plasma or serum; or the like. The sample can be applied to the methods of the invention directly or can be processed, extracted or purified to varying degrees before being used. The sample can be derived from a healthy subject or a subject suffering from a condition, disorder, disease or infection. For example, without limitation, the subject may be a healthy animal, e.g., human or non-human mammal, or an animal, e.g., human or non-human mammal, that has cancer, an inflammatory disease, autoimmune disease, metabolic disease, CNS disease, ocular disease, cardiac disease, pulmonary disease, hepatic disease, gastrointestinal disease, neurodegenerative disease, genetic disease, infectious disease or viral infection, or other ailment(s).

Preferably, protein mixtures derived from biological samples may be treated to deplete highly abundant proteins there from, in order to increase the sensitivity and performance of proteome analyses. By means of example, mammalian samples such as human serum or plasma samples may include abundant proteins, inter alia albumin, IgG, antitrypsin, IgA, transferrin, haptoglobin and fibrinogen, which may preferably be so-depleted from the samples. Methods and systems for removal of abundant proteins are known, such as, e.g., immuno-affinity depletion, and frequently commercially available, e.g., Multiple Affinity Removal System (MARS-7, MARS-14) from Agilent Technologies (Santa Clara, Calif.).

The term “protein peptide mixture” generally refers to a mixture of peptides derived from a protein or preferably from a mixture of two or more different proteins (i.e., protein mixture). The terms “peptide” or “protein peptide” as used herein generally refer to fragments of a protein derived by fragmentation of said protein or of any one or more of its polypeptide chains, into two or more fragments. While the terms encompass peptides of all sizes and molecular weights, peptides and protein peptide mixtures preferred in the invention may have average and/or median length of less than about 100 amino acids, e.g., less than about 90 amino acids, less than about 80 amino acids, more preferably less than about 70 amino acids or less than about 60 amino acids, even more preferably less than about 50 amino acids, e.g., particularly preferably less than about 40 amino acids or less than about 30 amino acids. In further embodiments, peptides and protein peptide mixtures preferred in the invention may have average and/or median length of at least about 5 amino acids, preferably at least about 10 amino acids, even more preferably at least about 15 amino acids, e.g., at least about 20 amino acids. Hence, in yet further embodiments, peptides and protein peptide mixtures preferred in the invention may have average and/or median length of between about 5 and about 100 amino acids, preferably between about 10 and about 50 amino acids, e.g., between about 10 and about 40 amino acids or between about 10 and about 30 amino acids. Such peptide sizes may be particularly amenable to proteome analysis.

### Pre-treatments

As noted, in the present methods the protein or protein mixture can be subjected to a pre-treatment, such as to desirably protect the N-terminal amino acid or the C-terminal amino acid in the protein or in proteins of the protein mixture. This desirably blocks said N-terminal amino acid or said C-terminal amino acid, such as to prevent their cleaving-off by the action of aminopeptidase or carboxypeptidase, respectively.

Suitable blocking reagents, as well as methods and conditions for attaching and detaching protecting groups will be clear to the skilled person and are generally described in standard handbooks of organic chemistry, such as “Protecting Groups”, P. Kocienski, Thieme Medical Publishers, 2000; Greene and Wuts, “Protective groups in organic synthesis”, 3rd edition, Wiley and Sons, 1999; incorporated herein by reference in their entirety.

Preferably, protection of the N-terminal amino acid can be achieved by suitably modifying the α-NH₂ group of said N-terminal amino acid. For example, said α-NH₂ group can be modified using reagents capable of selectively reacting with primary amino groups (“primary amino” alone or in combination refers to a group of formula —NH₂, optionally in any dissociation or protonation state such as —NH₃⁺) and presenting a non-reactive substituent for subsequent conditions. A blocking reagent may be generally substituted once or twice on each so-modified primary amine (i.e., —NH₂ gives —NHZ or —NZ₂, where Z is the substituent introduced by said blocking reagent).

In a non-limiting and preferred example, primary amines may be protected by acylation, e.g., acetylation, using reagents known per se, such as, e.g., using acetyl N-hydroxysulfosuccinimide, or may be protected using 2,4,6-trinitrobenzene sulfonic acid (TNBS), formaldehyde or any other group for reductive amination, ICPL (Isotope Coded Protein Labeling system from Serva Electrophoresis GmbH, Heidelberg, Germany) and the ITRAQ system (Applied Biosystems) reagents. Other suitable primary amino-modifying reagents have been extensively described in the art, for example, in Regnier et al. 2006 (Proteomics 6: 3968-3979). Reagents which introduce bulkier groups, whereby such groups can cause greater sterical hindrance for the action of aminopeptidase, may be more preferred. During modification of —NH₂ groups with acyl such as acetyl, the acyl moiety may be occasionally also introduced on the —OH group of Ser, Thr and/or Tyr. Such ester bonds are preferably subsequently broken by alkali hydrolysis at conditions that do not affect the acylation of the —NH₂ groups.

Preferably, protection of the C-terminal amino acid can be achieved by suitably modifying the α-COOH group of said C-terminal amino acid. For example, said α-COOH group can be modified using reagents capable of selectively reacting with carboxyl groups (“carboxyl” alone or in combination refers to a group of formula —COOH, optionally in any dissociation or protonation state such as —COO⁻) and presenting a non-reactive substituent for subsequent conditions.

In non-limiting and preferred examples, carboxyl groups may be protected by esterification to methyl esters, t-Butyl esters, benzyl esters, S-t-Butyl esters, or by conversion to 2-alkyl-1,3-oxazoline, to 5,6-dihydrophenanthridinamide or to hydrazide using reagents known per se (see, e.g., Greene and Wuts 1999, supra). Reagents which introduce bulkier groups, whereby such groups can cause greater sterical hindrance for the action of the carboxypeptidase, may be more preferred

Further advantageous pre-treatments of the protein mixture or protein peptide mixture may be included. For instance, Cys —SH groups in the protein, protein mixture or protein peptide mixture can be protected to avoid their reactivity, in particular oxidation, throughout the methods. Typically, the sample is first treated with a reducing agent known per se, such as, e.g., β-mercaptoethanol, dithiothreitol (DTT), dithioerythritol (DTE) or a suitable trialkylphosphine inter alia tris(2-carboxyethyl)phosphine (TCEP), to quantitatively reduce any oxidised —SH groups, e.g., disulphide bridges. The —SH groups are subsequently protected with a blocking reagent that reacts selectively with Cys side chains and presents a non-reactive substituent for subsequent conditions. By means of example and not limitation, —SH groups may be converted to acetamide derivatives by treatment with iodoacetamide in denaturing buffers (e.g., guanidium- or urea-containing buffers). Other blocking reagents, such as N-substituted maleimides (e.g., N-ethylmaleimide), acrylamide, N-substituted acrylamide or 2-vinylpyridine, may alternatively be used.

Pre-treatments may be applied simultaneously or sequentially in any suitable order. After and during pre-treatment, the sample may be optionally be purified using known techniques, such as solvent evaporation, washing, filtration, chromatographic techniques, etc.

### Fragmentation

A protein peptide mixture may be obtained by fragmentation of a protein or mixture of proteins, such as, e.g., by fragmentation of all or a fraction of proteins present in and/or isolated from a biological sample after the sample has been removed from biological source.

The term “fragmentation” as used herein in relation to a protein refers to cleavage, preferably enzymatic or chemical cleavage, of one or more peptide bonds within said protein or within any one or more of its polypeptide chains. Fragmentation of protein mixture denotes fragmentation of proteins constituting said protein mixture. Advantageously, proteins or protein mixtures may be fragmented so as to yield protein peptide mixtures having the preferred average or median chain lengths as detailed above.

When a protein or a polypeptide chain is cleaved at least at one peptide bond, such fragmentation generates a peptide that comprises the N-terminal end of said protein or polypeptide chain (“N-terminal peptide”) and a peptide that comprises the C-terminal end of said protein or polypeptide chain (“C-terminal peptide”). Where the protein or polypeptide chain is cleaved at two or more of its peptide bonds, such fragmentation additionally produces one or more peptides derived from the portion of the protein or polypeptide chain interposed between the parts corresponding to the N- and C-terminal peptides (“internal peptides”).

To ensure optimal characterisation of N-terminal or C-terminal peptides, it is desirable that fragmentation of individual molecules of a given protein occurs at the same peptide bond in substantially all individual molecules of said protein.

This can be advantageously achieved when the protein or protein mixture is fragmented preferentially at peptide bonds N-terminally or C-terminally adjacent to one or more specific amino acid residue types (denoted as X¹ . . . Xⁿ). The term “fragmented preferentially at” means that the fragmentation occurs substantially only at the recited peptide bond(s). Preferably, less than 10% of peptide bonds other than the recited ones would be cleaved, e.g., <7%, more preferably <5%, e.g., <4%, 3% or <2%, most preferably <1%, e.g., <0.5%, <0.1%, or <0.01%.

Preferably, a protein or protein mixture will be fragmented at substantially all recited peptide bonds. Hence, the fragmentation would occur substantially quantitatively at peptide bonds N-terminally or C-terminally adjacent to residues of the one or more types X¹ . . . Xⁿ.

To achieve a protein peptide mixture displaying preferred average and/or median peptide lengths, the protein or protein mixture may be advantageously fragmented adjacent to a relatively small number of amino acid residue types X¹ . . . Xⁿ, such as at peptide bonds adjacent to 5 or less amino acid residue types (i.e., n≧5), more preferably n≧4, even more preferably n≧3, still more preferably n≧2, or preferably at peptide bonds adjacent to only 1 amino acid residue type (i.e., n=1).

The one or more specific amino acid residue types X¹ . . . Xⁿ adjacent to which fragmentation is contemplated herein may be selected from any amino acid residues, including but not limited to amino acids found in naturally occurring proteins, amino acids carrying a co- or post-translational modification, amino acids including a non-natural isotope, or amino acids further chemically and/or enzymatically altered prior to the fragmentation, etc.

A suitable frequency of cleavage may be preferably achieved when the fragmentation takes place adjacent to one or more of the 20 common amino acid residue types found in natural proteins and/or adjacent to one or more of residue types obtained from any of the 20 common amino acid residue types by suitable modification of the starting proteins. Accordingly, in a preferred embodiment, the protein or mixture of proteins is fragmented preferentially at peptide bonds adjacent to one or more amino acid residue types X¹ . . . Xⁿ chosen from the group consisting of: Gly, Pro, Ala, Val, Leu, Ile, Met, Cys, Phe, Tyr, Trp, His, Lys, Arg, Gln, Asn, Glu, Asp, Ser and Thr; optionally including a co- or post-translational modification, a chemical and/or enzymatic alteration prior to the fragmentation, or including a non-natural isotope, etc.

Fragmentation may be effected by suitable physical, chemical and/or enzymatic agents, more preferably chemical and/or enzymatic agents, even more preferably enzymatic agents, e.g., proteinases, preferably endoproteinases. Preferably, the fragmentation may be achieved by one or more, preferably one, endoproteinase, i.e., a protease cleaving internally within a protein or polypeptide chain (i.e., endoproteolytic cleavage or fragmentation). A non-limiting list of suitable endoproteinases includes serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases.

By means of example not limitation, protein fragmentation may be achieved using trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain). The invention encompasses the use of any further known or yet to be identified enzymes; a skilled person can choose suitable protease(s) on the basis of their cleavage specificity and the frequency of occurrence of the amino acid(s) adjacent to which fragmentation is induced, to achieve desired protein peptide mixtures.

In a preferred embodiment, the fragmentation may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, etc. Trypsin is particularly useful in proteomics applications, inter alia due to high specificity (C-terminally adjacent to Arg and Lys except where the next residue is Pro) and efficiency of cleavage. The invention also contemplates the use of any trypsin-like protease, i.e., with a similar specificity to that of trypsin.

It has been suggested that some aminopeptidases may cleave-off N-terminal proline with reduced efficiency. Although not observed by the present inventors, this might in theory lead to incomplete hydrolysis of unwanted peptides containing Pro. To avoid this, fragmentation of proteins to protein peptide mixtures may be advantageously performed using a prolyl endopeptidase (EC 3.4.21.26), i.e., endopeptidase that specifically cleaves C-terminally to Pro, such as by example but without limitation the recombinant Pro-C endopeptidase available from Fluka (Cat. No. 45167). Hereby, Pro would become the ultimate residue of unwanted peptides, which would therefore be completely hydrolysed by aminopeptidase.

In other embodiments, chemical reagents may be used. By means of example and not limitation, CNBr can fragment proteins at Met; BNPS-skatole can fragment at Trp.

The conditions for treatment, e.g., protein concentration, enzyme or chemical reagent concentration, pH, buffer, temperature, time, can be determined by the skilled person depending on the enzyme or chemical reagent employed.

### Exopeptidases

Methods of the invention employ exopeptidases, namely aminopeptidases or carboxypeptidases, to hydrolyse unwanted unprotected peptides, thus leaving behind and enriching for desired protected N-terminal or C-terminal peptides, respectively.

As used herein, the term “exopeptidase” refers to a hydrolase enzyme which hydrolyses the peptide bonds adjacent to terminal amino acids of a peptide or protein, thereby removing said terminal amino acids from said peptide or protein.

The term “aminopeptidase” refers to an exopeptidase which hydrolyses the peptide bond adjacent to the N-terminal amino acid of a peptide or protein, thereby releasing said N-terminal amino acid from said peptide or protein. Exemplary but non-limiting of aminopeptidases are grouped under EC classification numbers EC 3.4.11.1 to EC 3.4.11.23. Aminopeptidases as used herein may encompass inter alia naturally occurring aminopeptidases (e.g., as isolated from natural source or recombinantly produced), as well as engineered aminopeptidases (such as, e.g., derived by modification of naturally occurring aminopeptidases) such as to obtain optimal or evolved enzymatic characteristics for progressive removal of unprotected amino acids.

The term “carboxypeptidase” refers to an exopeptidase which hydrolyses the peptide bond adjacent to the C-terminal amino acid of a peptide or protein, thereby releasing said C-terminal amino acid from said peptide or protein. Exemplary but non-limiting carboxypeptidases are grouped under EC classification numbers EC 3.4.16 (serine-type carboxypeptidases), EC 3.4.17 (metallocarboxypeptidases) and EC 3.4.18 (cysteine-type carboxypeptidases). Carboxypeptidases as used herein may encompass inter alia naturally occurring carboxypeptidases (e.g., as isolated from natural source or recombinantly produced), as well as engineered carboxypeptidases (such as, e.g., derived by modification of naturally occurring carboxypeptidases) such as to obtain optimal or evolved enzymatic characteristics for removal of amino acids.

In an embodiment, an aminopeptidase or carboxypeptidase may display substantially no preference or specificity for the type of amino acid that it cleaves-off, such that it would successively remove all amino acid types from a peptide's N-terminus or C-terminus, respectively, thereby completely hydrolysing the peptide. Non-limiting examples of non-specific aminopeptidases include inter alia aminopeptidase I from Streptomyces griseus (Spungin & Blumberg 1989, Eur J Biochem 183: 47; EC 3.4.11.22, #A9934 Sigma Aldrich), Microsomal aminopeptidase M from Sus scrofa (EC 3.4.11.2, #L5006 Sigma Aldrich), Aeromonas proteolytica aminopeptidase (EC 3.4.11.10, #A8200 Sigma Aldrich), and porcine leucine aminopeptidase (EC 3.4.11.1). Non-limiting examples of non-specific carboxypeptidases include inter alia carboxypeptidase C and Y (EC 3.4.16.5), and Carboxypeptidase P.

In another embodiment, the methods may employ aminopeptidases or carboxypeptidases that display preference or specificity for cleaving-off one or more particular amino acid types. In this embodiment, to achieve successive release of all amino acid types from a peptide's N-terminus or C-terminus, combinations of two or more aminopeptidases with complementary specificities or of two or more carboxypeptidases with complementary specificities, respectively, may be used. By means of example and not limitation, the combination of prolyl aminopeptidase (EC 3.4.11.5 removing N-terminal prolines) with Aminopeptidase M (EC 3.4.11.2) can compensate for the delayed activity on N-terminal prolines.

Aminopeptidases or carboxypeptidases for use herein may be isolated as known in the art from a variety of respective sources, and also include any recombinantly produced forms thereof.

The conditions for peptide hydrolysis, e.g., peptide concentration, exopeptidase concentration, pH, buffer, temperature, time, post-reaction inactivation, etc., can be determined by the skilled person depending on the enzyme employed.

### Separation of N-terminal or C-terminal Peptides

Depending on parameters such as the complexity of the protein sample, the N-terminal or C-terminal peptides isolated as above can be directly subjected to methods for peptide identification, or may be further resolved (fractionated) using a single- or multi-dimensional separation process prior to such identification.

In a “single-dimensional” separation process a sample of analytes (peptides) is subjected to a single separation step which resolves analytes on the basis of one or more, such as one, physical and/or chemical property. In a “multi-dimensional” separation process a sample of analytes is subjected to a sequence of two or more separation steps (“dimensions”), each of which acts upon all or a part of analytes separated in a previous separation step, wherein any two analytes resolved in a given separation step remain resolved in subsequent separation steps, and wherein the distinct separation steps resolve analytes on the basis of different physical and/or chemical properties. Preferably, the distinct separation steps are orthogonal, such that peptides not resolved (i.e., recovered in same fraction) in one step will be resolved in another step. Typically, to realise a multidimensional separation, any or all fractions from a given separation step are each individually resolved in a subsequent separation step.

Analytical separation methods that can fractionate peptides on the basis of one or more physical and/or chemical properties are well-known in the art.

For example, electrophoresis applications exist to resolve peptides on the basis of net charge, EPM or pI, including inter alia gel electrophoresis such as capillary gel electrophoresis (CGE), capillary zone electrophoresis (CZE), free flow electrophoresis (FFE), isoelectric focusing (IEF) including capillary isoelectric focusing (CIEF), isotachophoresis (ITP), capillary electrochromatography (CEC), and the like.

For example, size exclusion chromatography (SEC) including gel filtration chromatography or gel permeation chromatography may be applied to resolve peptides based on molecular size.

In a particularly preferred example, peptides may be resolved by chromatography, preferably 1 D- or 2D-chromatography. The term “chromatography” includes methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Exemplary types of chromatography useful herein include, without limitation, high-performance liquid chromatography (HPLC), normal phase HPLC (NP-HPLC), reversed phase HPLC (RP-HPLC), ion exchange chromatography, such as cation or anion exchange chromatography, hydrophilic interaction chromatography (HILIC), hydrophobic interaction chromatography (HIC), affinity chromatography such as immuno-affinity and immobilised metal affinity chromatography. While particulars of these chromatography types are well known in the art, for further guidance see, e.g., Meyer M., 1998, ISBN: 047198373X and Cappiello et al. 2001 (Mass Spectrom Rev 20: 88-104), incorporated herein by reference.

Preferably, the chromatography may employ liquid mobile phase (i.e., liquid chromatography). Also preferably, the chromatography may be columnar, i.e., wherein the stationary phase is deposited or packed in a column. In yet further preferred embodiment, the chromatography is HPLC, such as preferably RP-HPLC. Columns and conditions for performing HPLC separations including RP-HPLC are generally known to the skilled person, and described in, e.g., Practical HPLC Methodology and Applications, Bidlingmeyer, B. A., John Wiley & Sons Inc., 1993.

### Identification and Quantification of Peptides and Proteins

The methods and systems of the invention find particular use in proteomics applications. The N-terminal or C-terminal peptides isolated and optionally fractionated as above are highly representative of and can thus identify the corresponding proteins in a starting sample.

In a preferred approach, further separation, analysis and/or identification of the peptides may be performed using a mass spectrometer. Otherwise, said peptides may be analysed and/or identified using other methods such as, e.g., activity measurement in assays, analysis with specific antibodies, Edman sequencing, etc.

In an embodiment, N-terminal or C-terminal peptides released from the isolation or separation process can be directly (on-line) fed to an analyser (e.g., on-line LC/MS/MS). Otherwise, the peptides resolved by the separation process may be collected in fractions which, optionally following additional manipulation (e.g., concentration and/or spotting onto a MALDI-target; or advantageously, mixing with matrix in a microtee prior to deposition on MALDI targets, thereby eliminating the need for concentration and manual spotting; etc.), can be fed to an analyser.

Preferably, the peptides are analysed and identified using mass spectrometry (MS), preferably high-throughput MS techniques known per se that can obtain precise information on the mass and preferably also on (partial) amino acid sequence of the peptides (e.g., in tandem mass spectrometry, MS/MS; or in post source decay TOF MS). Such information can be used in database searching to trace the peptides back to their parent proteins.

MS arrangements and instruments appropriate for peptide analysis are commonly known and may include, without limitation, matrix-assisted laser desorption/ionisation time-of-flight (MALDI-TOF) MS systems; MALDI-TOF post-source-decay (PSD) systems; MALDI-TOF/TOF systems; electrospray ionisation (ESI) 3D or linear (2D) ion trap MS systems; ESI triple quadrupole MS systems; ESI quadrupole orthogonal TOF systems (Q-TOF); or ESI Fourier transform MS systems; etc. Peptide ion fragmentation in tandem MS (MS/MS) may be achieved using manners established in the art, such as, e.g., collision induced dissociation (CID).

Algorithms and software exist in the art that compare experimental mass spectra and optionally also (partial) sequence information for the analysed peptides with a database of peptide masses/sequences predicted on the basis of sequence information in protein and nucleic acid databases, and identify the corresponding peptides: e.g., ProFound, X! Tandem, (http://prowl.rockefeller.edu), MASCOT (http://www.matrixscience.com, Matrix Science Ltd. London), Sequest (http://fields.scripps.edu/sequest/; U.S. Pat. No. 6,017,693; U.S. Pat. No. 5,538,897), OMSSA (http://pubchem.ncbi.nlm.nih.gov/omssa/), etc. Starting from the known identity of so-detected peptides, the corresponding proteins can be easily found by sequence database searching using these or other software tools. Identification of N-terminal peptides can also benefit from the use of specialised N-terminally ragged databases to account for protein processing, as known in the art (e.g., Gevaert et al. 2003. Nat Biotechnol 21: 566-569; Martens et al. 2005. Proteomics 5: 3139-3204).

Generally, the herein disclosed methods may achieve identification of any number or even substantially all (i.e., comprehensive analysis) N-terminal or C-terminal peptides present in starting protein peptide mixtures. Optionally, the methods may further encompass art established technique(s) to determine the relative or absolute quantity of one or more proteins in the starting sample (see, e.g., WO 03/016861, WO 02/084250 or WO 2004/111636).

In a preferred embodiment, the methods and systems of the present invention may be employed to identify proteins differentially present between samples, preferably biomarkers.

“Marker” or “biomarker” as used herein refer to a protein or polypeptide which is differentially present in a sample taken from subjects having a genotype or phenotype of interest and/or who have been exposed to a condition of interest (herein “query sample”), as compared to an equivalent sample taken from control subjects not having said genotype or phenotype and/or not having been exposed to said condition (herein “control sample”). Samples can be as disclosed above and may be broadly applied to compare for instance subcellular fractions, cells, tissues, biological fluids (e.g., nipple aspiration fluid, saliva, sperm, cerebrospinal fluid, urine, blood, serum, plasma, synovial fluid), organs and/or complete organisms.

A particularly relevant phenotype may be a pathological condition of interest in patients, such as, e.g., cancer, an inflammatory disease, autoimmune disease, metabolic disease, CNS disease, ocular disease, cardiac disease, pulmonary disease, hepatic disease, gastrointestinal disease, neurodegenerative disease, genetic disease, infectious disease or viral infection; vis-à-vis the absence of such condition in healthy controls. Other comparisons may be envisaged between samples from, e.g., stressed vs. non-stressed conditions/subjects, drug-treated vs. non drug-treated conditions/subjects, benign vs. malignant diseases, adherent vs. non-adherent conditions, infected vs. uninfected conditions/subjects, transformed vs. untransformed cells or tissues, different stages of development, conditions of overexpression vs. normal expression of one or more genes, conditions of silencing or knock-out vs. normal expression of one or more genes, and so on.

The phrase “differentially present” refers to a demonstrable, preferably statistically significant, difference in the quantity and/or frequency of a protein or polypeptide (also including endogenously proteolytically processed forms thereof) in query samples as compared to control samples. For example, a marker may be a protein which is present at an elevated level or at a decreased level in query samples compared to control samples. A marker may also be a protein which is detected at a higher frequency or at a lower frequency in query samples compared to control samples.

For example, a protein may be differentially present between two samples if the protein's quantity in one sample is at least about 120%, at least about 130%, at least about 150%, at least about 180%, at least about 200%, at least about 300%, at least about 500%, at least about 700%, at least about 900% or at least about 1000% of its quantity in the other sample; or if it is detectable in one sample but not detectable in the other sample.

Otherwise, a protein may be differentially present between two sets of samples if the frequency of detecting the protein in one set of samples is at least about 120%, at least about 130%, at least about 150%, at least about 180%, at least about 200%, at least about 300%, at least about 500%, at least about 700%, at least about 900% or at least about 1000% of the frequency of detecting the protein in the other set of samples; or if the protein is detectable at a given frequency in one set of samples but is not detected in the second set of samples.

Hence, analysis of N-terminal or C-terminal peptides sorted as herein can identify proteins differentially present between query and control samples, thereby identifying potential biomarkers.

In an embodiment, query samples and control samples may be analysed separately and abundances of corresponding peptides may be subsequently compared there between. This is generally known in the art as label-free profiling.

Preferably, to reduce variance between the to-be-compared samples, the samples may be analysed in the same sorting and separation experiment insofar peptides derived from such samples are differentially labelled allowing to attribute a given readout to one of the starting samples. For example, samples (typically two samples) can be treated so that peptides derived from one sample contain one isotope and peptides obtained from the other sample contain another isotope of the same element. Such differentially-labelled samples may be analysed in the same sorting and separation experiment. The mass difference caused by the presence of other isotopes allows to distinguish—and compare the relative intensity of—peaks corresponding to equivalent peptides from the differentially-labelled samples on MS.

Hence, in an embodiment the protein peptide mixture (“PPM”) to be analysed may be prepared by combining, preferably in equal amounts:
a first protein peptide mixture (“PPM1”) derived from a first sample (e.g., a query sample), the peptides of mixture PPM1 being labelled with a first isotope; and
a second protein peptide mixture (“PPM2”) derived from a second sample (e.g., a control sample), the peptides of mixture PPM2 being labelled with a second isotope different from the first isotope.

After isolating, resolving and analysing the N-terminal or C-terminal peptides of the protein peptide mixture, one or more N-terminal or C-terminal peptides differentially present between the first and second samples can be identified by comparing the peak heights or areas of identical but differentially isotopically labelled peptides. The identity of the isolated peptide and its corresponding protein—potentially representing a biomarker—can then be determined. Here above, the abbreviations “PPM”, “PPM1” and “PPM2” are merely intended to assist perusal of the specification, and carry no actual connotations.

The differential isotopic labelling of peptides in the first and second samples can be done in many art-known ways. A key element is that a particular peptide originating from the same protein in a first and second samples is identical, except for the presence of a different isotope in one or more amino acids of the peptide. Examples of pairs of distinguishable isotopes are ¹²C and ¹³C, ¹⁴N and ¹⁵N or ¹⁶O and ¹⁸O. Peptides labelled with such isotopes are chemically very similar, separate chromatographically in the same manner and also ionise in the same way. However, when fed into an analyser, such as MS, they will segregate into the distinguishable light and heavy peptide. The results of the mass spectrometric analysis of isolated peptides will thus be a plurality of pairs of closely spaced twin peaks, each twin peak comprising a heavy and the corresponding light peptide. The ratios (relative abundance) of the peak intensities of the heavy and light peak in each pair are then measured. These ratios give a measure of the relative amount (differential presence) of that peptide (and its corresponding protein) in each sample. The peak intensities can be calculated in a conventional manner (e.g., by calculating the peak height or peak surface).

Incorporation of isotopes into peptides can be obtained in multiple ways. In one approach proteins are labelled by growing cells in media supplemented with an amino acid containing the different isotopes (SILAC; see, e.g., in Ong et al. 2002 (Mol Cell Proteomics 1(5): 376-86)).

In a preferred embodiment, the different isotopes can be incorporated by an enzymatic approach. For instance, labelling can be carried out by treating one sample comprising proteins with trypsin in H₂¹⁶O and the second sample comprising proteins with trypsin in H₂¹⁸O. Trypsin incorporates two oxygens of water at the COOH-termini of the newly generated sites during cleavage. Alternatively, treating protein peptide mixture post-digestion with trypsin in H₂¹⁶O or H₂¹⁸O leads to incorporation of two oxygen atoms (¹⁶O or ¹⁸O, respectively) at the COOH-termini of the component peptides (see, e.g., US 2006/105415), except the C-termini of the original proteins.

Having identified suitable biomarkers, the methods of the invention may also be employed in a diagnostic mode to detect the presence, absence or a variation in expression level of one or more biomarkers or a specific set of proteins indicative of a disease state (e.g., such as cancer, neurodegenerative disease, inflammation, cardiovascular diseases, viral infections, bacterial infections, fungal infections or any other disease) in a sample.

### EXAMPLES

### Example 1

### Acetylation Protects Peptides from Degradation by Aminopeptidase

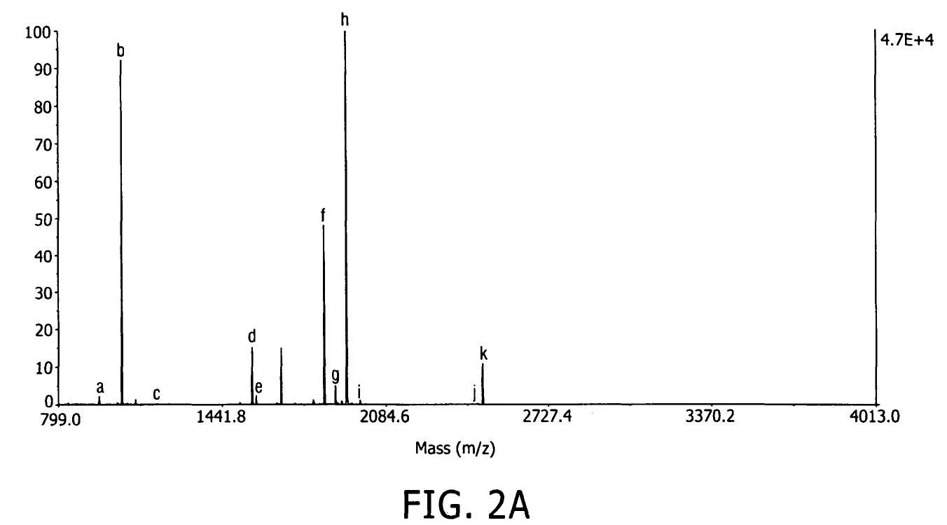

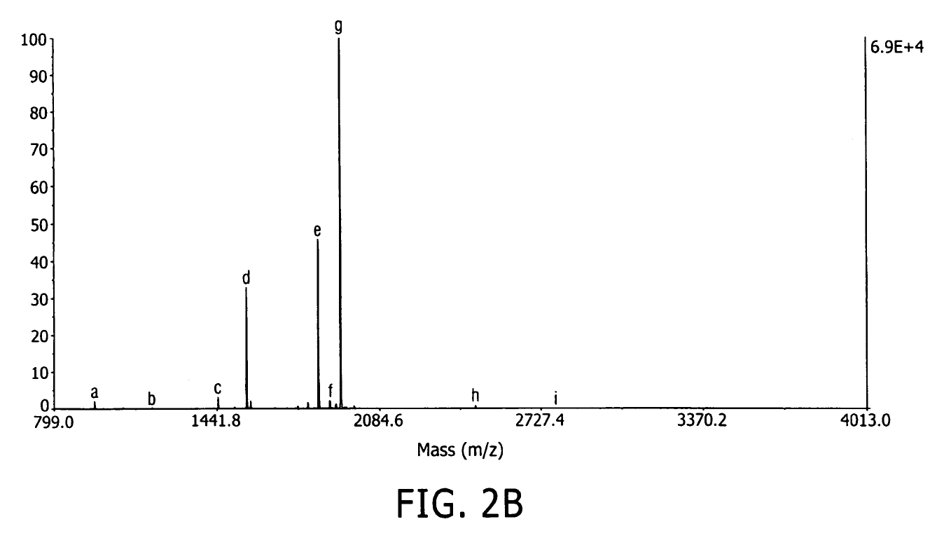

In the first experiment we investigated the effect of using acetylation as N-terminal blocking on progressive hydrolysis of peptides by bacterial aminopeptidase. As substrate for aminopeptidase activity we used the unprotected peptides from the Pepmix4 calibration mix (PepMix4, LaserBioLabs #C104). One tube was dissolved in 100 μl water resulting in peptide concentrations ranging form 8-50 pmoles/μl. As protected peptides we used 2 acetylated peptides designated P1380 and P1384 (for sequences see table 1). Both were diluted to 40 pmoles/μl. The sample reaction mixture that was used contained 1 μl of PepMix4 and 1 μl of each P1380 and P1384, all added to 20 mM TrisHCl buffer pH8, to a final volume of 20 μl. One sample was incubated overnight at 37° C. with 1 unit of Aeromonas proteolytica aminopeptidase (Sigma-aldrich, A8200) while another sample was left untreated. The reaction was stopped with 10% Trifluoroacetic acid. After incubation, 11 μl of the sample was used for purification by Perfectpure C-18 tips (Eppendorf 957 01 002-4). The purified sample was mixed with an equal volume of CHCA MALDI matrix solution (LaserBioLabs) and spotted on MALDI target plates. MALDI measurements for the 2 experiments are shown in FIG. 2. From these studies it is clear that acetylation protects peptides from hydrolysis by bacterial aminopeptidase. The bacterial aminopeptidase can thus be used to remove unprotected internal peptides while not affecting the blocked peptides.

### Example 2

### ICPL Leads to N-terminal Protection from Aminopeptidase Activity

We investigated whether other blocking groups could improve the protection from aminopeptidase activity. In this experiment we also studied the use of Aminopeptidase M purified form pig kidney microsomes.

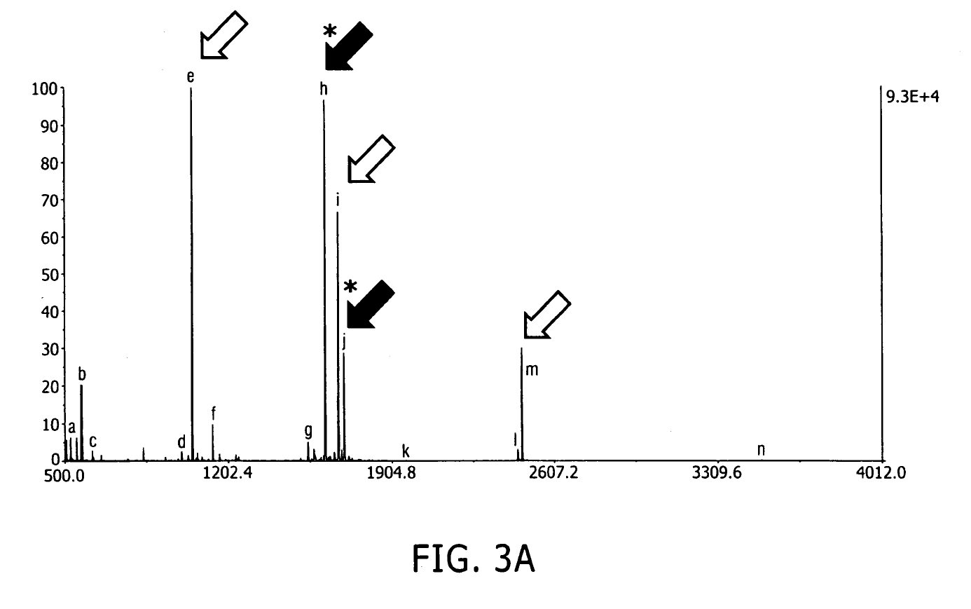

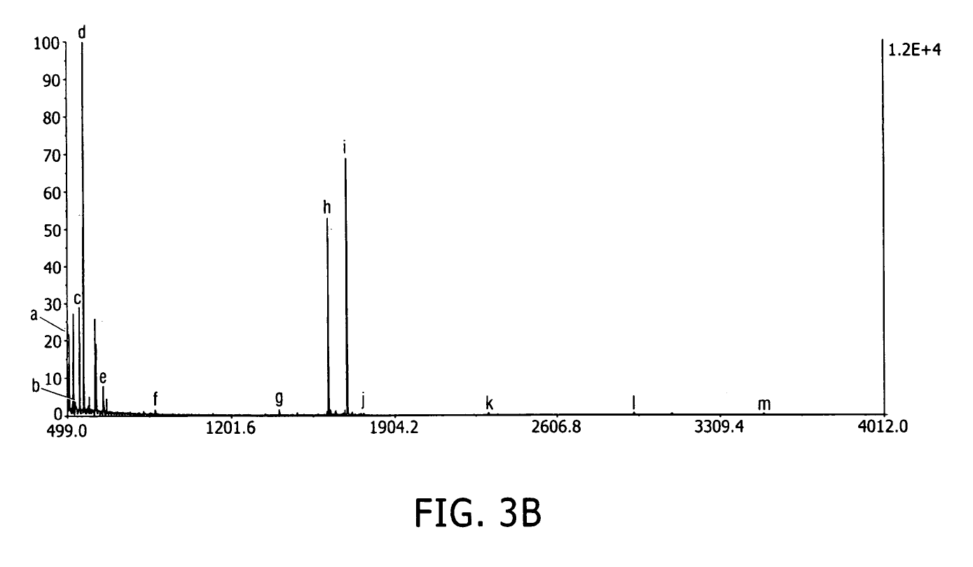

To prevent contamination issues with the commercial aminopeptidase M preparation (Sigma Aldrich, #L5006), the enzyme batch was dialyzed against 60 mM Sodiumphosphate at pH7. The ICPL reagent (Serva ICPL™-kit, #39230) was employed as a blocking agent to improve the protection from aminopeptidase activity. The rationale behind this is the greater sterical hindrance for enzyme activity if a bigger group is introduced on the N-terminus. ICPL labeling of 4 peptides designated L2 to L5 (Table 1), was performed according to the manufacturer's instructions. A mixture was prepared with 200 pmoles of the Pepmix4 peptides, 50 pmoles of each L2 to L5 and 0.05U of the dialyzed aminopeptidase M. An overnight incubation at 37° C. was performed. The reaction was stopped with 2 μl 10% TFA. Peptides were purified by Perfectpure tips and subsequently spotted on MALDI targets. The MALDI mass spectra are shown in FIG. 3, showing that N-terminal ICPL modification can suitably protect.

TABLE 1
Sequences of peptides used in the examples

| | |
|---|---|
| P1380 | AcIPMYSIITPNVLR |
| P1384 | AcSELEEDIIPEEDIISR |
| L2 | LADGGATNQGR |
| L3 | ELSEALGQIFDSQR |
| L4 | STHTLDLSR |
| L5 | GLNLTEDTYKPR |

### Example 3

### The Modified N-terminal Peptides of Proteins are more Resistant to Aminopeptidase Activity when Compared to their Internal Peptides

This experiment was performed to show that the N-terminal peptides can be enriched by their altered susceptibility to aminopeptidase activity.

For this analysis, we prepared a mixture of 7 proteins (α-1-antitrypsin, hemoglobin, transferrin, albumin, β-lactoglobulin, α-1-acid glycoprotein and catalase; all from Sigma-aldrich) in PBS containing 4M Guanidine HCl. A total of 0.7 mg protein (0.1 mg/protein) was used for sample preparation. The sample was first reduced by treatment for 10′ at 30° C. with 6 μl 0.1M Tris(2-carboxyethyl)-phosphine Hydrochloride (TCEP.HCl, Pierce, #20490) after adjusting to pH 7.0. After this reduction, the free sulfhydryl groups on cysteines are alkylated by adding 6 μl 0.2M iodoacetamide (Fluka, #57670) for 60′ at 30° C. The sample was subsequently brought on a NAP™5 gelfiltration column (GE healthcare, #17-0853-02) to remove excess reagent and to transfer the sample to 50 mM sodiumphosphate pH8 with 1.4 M Guanidine HCl. Sulfo-NHS acetate is then added (10 μl of 50 mg/ml, Sulfosuccinimidyl Acetate, Pierce, #26777) for 90′ at 30° C. Acetylation events on serine or threonine are reversed by subsequent addition of 0.4 μl 50% hydroxylamine solution. Excess reagent is again removed by introduction of a gelfiltration step (PD-10 columns, GE Healthcare, #17-0851-01) with buffer exchange to 50 mM ammoniumbicarbonate. The volume was reduced by vacuum centrifugation to 2 ml. Each vial of 1 ml sample was digested overnight at 37° C. with 10 μg Sequencing Grade Modified Trypsin (Promega) after 5′ incubation at 99° C.

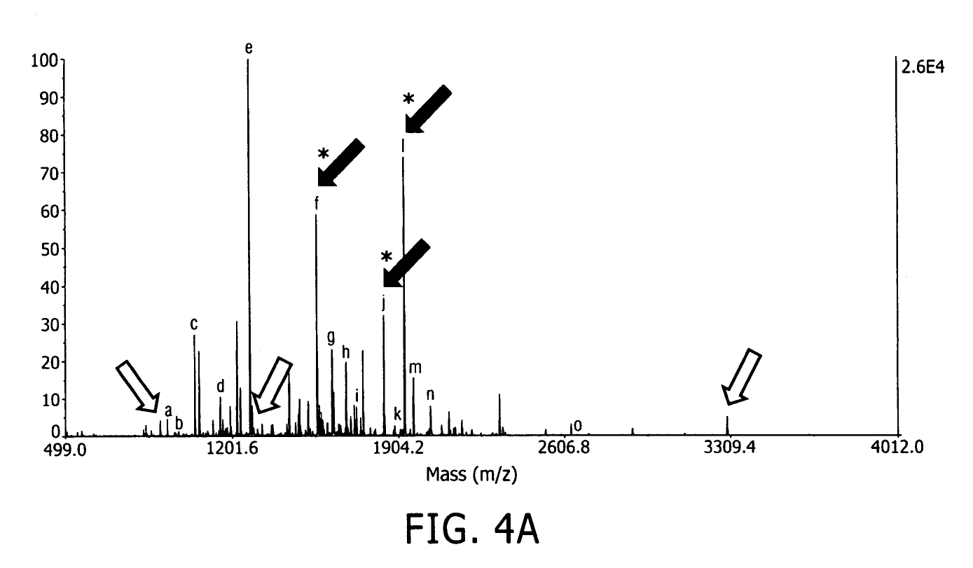

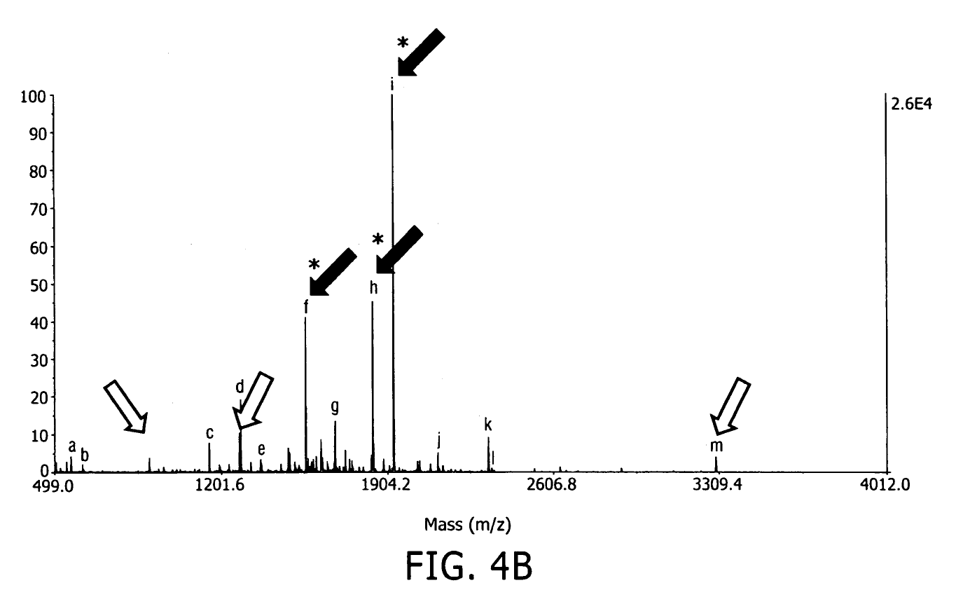

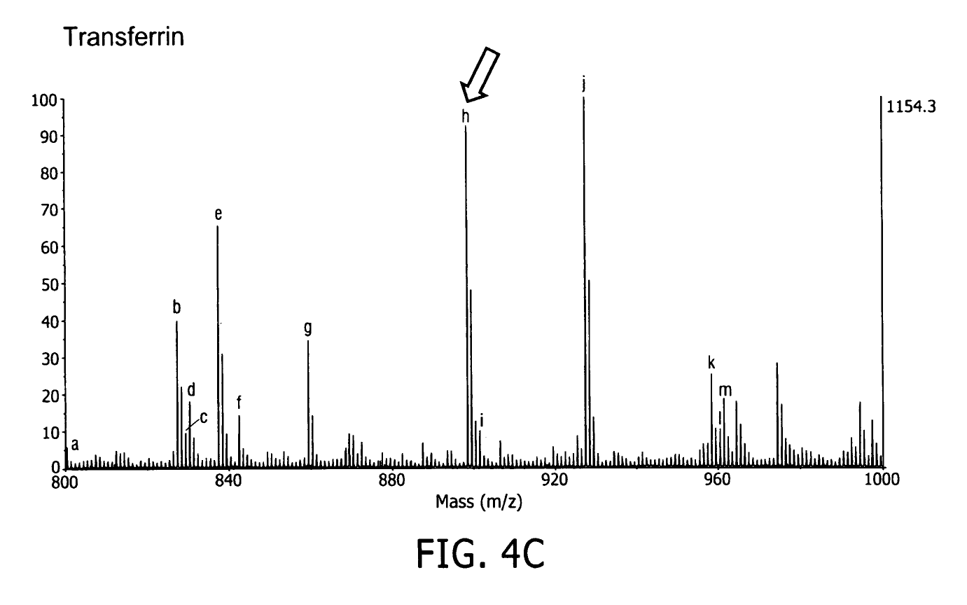

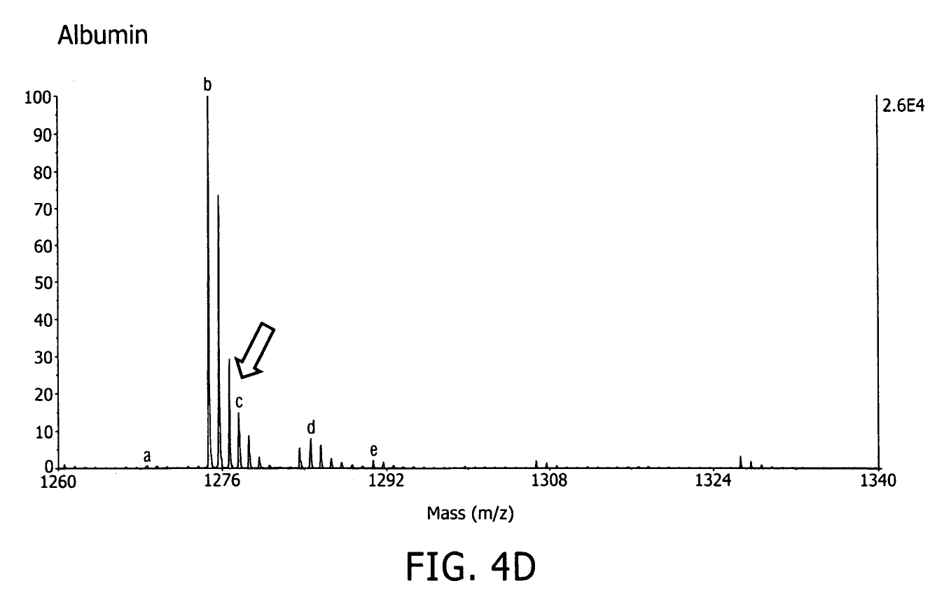

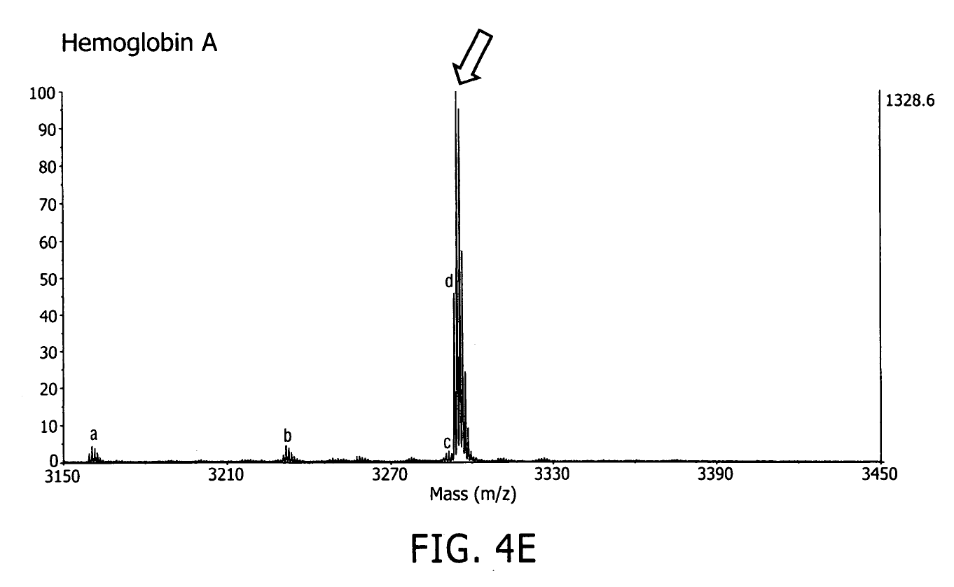

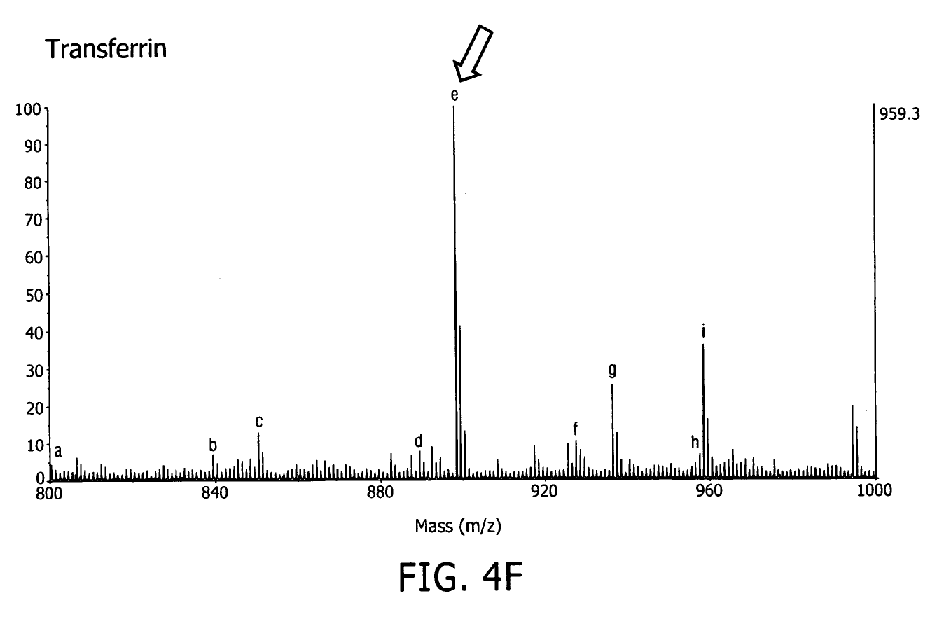

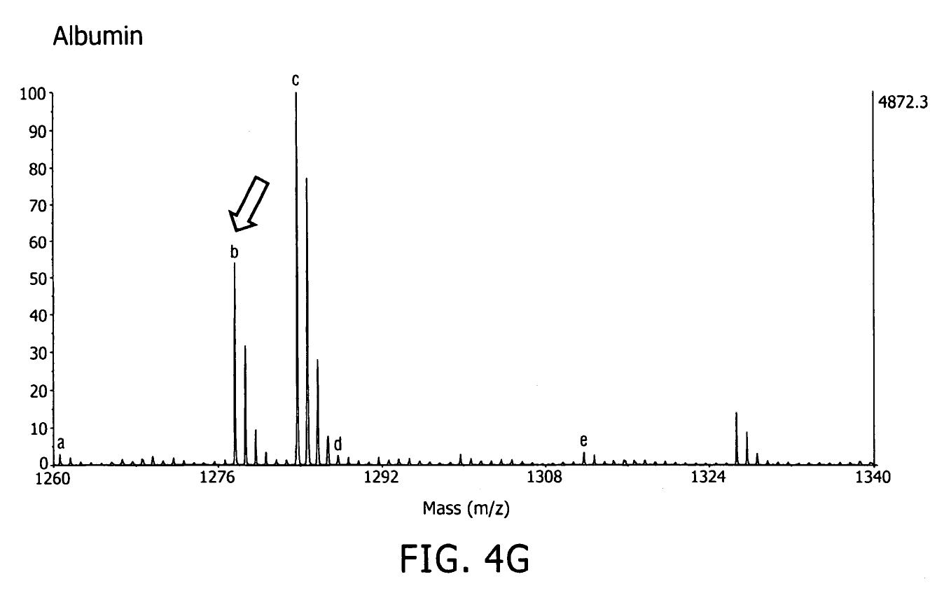

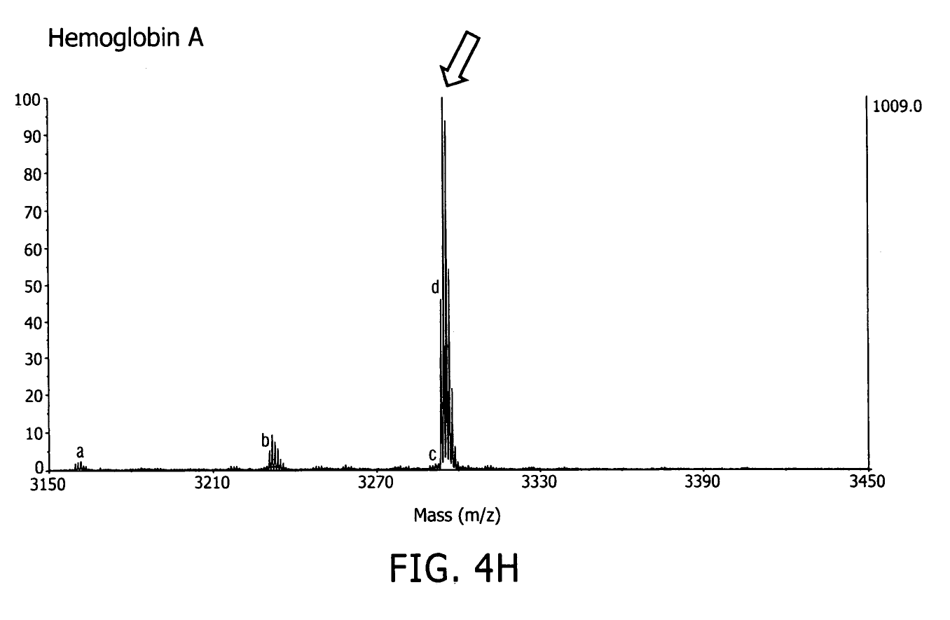

A mixture was prepared wherein 1 μl (40 pmoles) of the acetylated P1380 and P1384 peptides were added to 10 μl of the peptide digest described higher. This was incubated for 5′ at 99° C. again to inactivate trypsin prior to the experiment. A ‘sequence grade’ aminopeptidase M preparation (Sigma-aldrich L9776) was added in a final volume of 20 μl. The mixture of peptides from the 7 proteins and the acetylated peptides are incubated for 5′ at room temperature with the aminopeptidase, or were left untreated. The reaction was stopped by addition of 2 μl 10% Trifluoroacetic acid. An untreated sample was run in parallel as a reference. The samples were purified using Perfectpure tips and spotted on MALDI targets. The MALDI analysis of these samples is shown in FIG. 4. These experiments clearly demonstrate altered hydrolysis rates of N-terminal peptides as shown by the identification of the expected masses in the aminopeptidase treated sample. The spiked acetylated peptides are also protected from aminopeptidase activity. The results support the fact that this approach can be used to obtain an N-terminal signature.

### Tables

The tables below list values represented by alphabetical characters in FIGS. 2A, 2B, 3A, 3B, and 4A-4H. The character ‘n’ in a value represents an unspecified digit.

FIG. 2A

| | |
|---|---|
| a | 958.44940 |
| b | 1046.49426 |
| c | 1184.61560 |
| d | 1558.80225 |
| e | 1574.78809 |
| f | 1841.82886 |
| g | 1886.85010 |
| h | 1928.85706 |
| i | 1982.75647 |
| j | 2448.18018 |
| k | 2465.09766 |

FIG. 2B

| | |
|---|---|
| a | 958.43988 |
| b | 1184.60852 |
| c | 1446.68298 |
| d | 1558.79407 |
| e | 1841.82227 |
| f | 1886.84290 |
| g | 1928.85315 |
| h | 2465.08618 |
| i | 2756.15356 |

FIG. 3A

| | |
|---|---|
| a | 524.145(S585) |
| b | 573.315(S2020) |
| c | 622.054(S99) |
| d | 1001.522(S301) |
| e | 1046.543(S12255) |
| f | 1134.550(S915) |
| g | 1543.875(S557) |
| h | 1616.771(S14893) |
| i | 1672.917(S13548) |
| j | 1697.799(S6664) |
| k | 1960.923(S30) |
| l | 2448.194(S1964) |
| m | 2465.198(S23673) |
| n | 3494.652(S220) |

FIG. 3B

| | |
|---|---|
| a | 506.118(S403) |
| b | 528.173(S34) |
| c | 550.110(S556) |
| d | 568.119(S1932) |
| e | 652.327(S156) |
| f | 874.431(S53) |
| g | 1405.727(S167) |
| h | 1616.803(S6407) |
| i | 1697.854(S8959) |
| j | 1768.898(S80) |
| k | 2309.192(S94) |
| l | 2934.501(S96) |
| m | 3494.834(S48) |

FIG. 4A

| | |
|---|---|
| a | 927.46521(A4293, R15104, S326) |
| b | 974.47803(A1312R15530, S89) |
| c | 1043.57043(A27441, R15714, S1524) |
| d | 1150.5nnn0(A11025, R15867, S444) |
| e | 1274.69116(A120275, R16367, S3684) |
| f | 1558.82739(A80051, R16267, S1849) |
| g | 1623.84644(A34017, R14963, S466) |
| h | 1681.90710(A24690, R17619, S673) |
| i | 1742.76660(A6232, R17691, S158) |
| j | 1841.85974(A47339, R16172, S1176) |
| k | 1921.79749(A2655, R15079, S72) |
| l | 1928.89392(A122711, R15326, S3128) |
| m | 1965.89319(A21960, R15650, S648) |
| n | 2038.86511(A13177, R14486, S248) |
| o | 2633.26221(A38818, R14361, S632) |

FIG. 4B

| | |
|---|---|
| a | 568.12177(A3319, R12874, S562) |
| b | 622.03845(A542, R13828, S99) |
| c | 1150.61438(A7486, R16682, S774) |
| d | 128n.71399(A21495, R16483, S1618) |
| e | 1367.70813(A4344, R14646, S132) |
| f | 1558.87451(A51393, R16959, S2099) |
| g | 1681.95886(A16146, R16803, S628) |
| h | 1841.91919(A68957, R15717, S2270) |
| i | 1928.95471(A174148, R14083, S5849) |
| j | 2116.18335(A6652, R15368, S354) |
| k | 2331.18677(A16270, R14272, S751) |
| l | 2344.22314(A2058, R11839, S183) |
| m | 3230.65479(A511, R10493, S91) |

FIG. 4C

| | |
|---|---|
| a | 800.35004(A343, R14457, S20) |
| b | 827.41223(A1636, R14639, S165) |
| c | 829.39880(A656, R8829, S31) |
| d | 830.38599(A1009, R11423, S68) |
| e | 837.44696(A2782, R14188, S269) |
| f | 842.47925(A757, R13310, S55) |
| g | 859.47003(A1561, R14238, S126) |
| h | 898.47131(A4054, R14517, S316) |
| i | 901.47656(A512, R15148, S22) |
| j | 927.46521(A4293, R15104, S326) |
| k | 958.45306(A1215, R14594, S71) |
| l | 960.52008(A660, R11169, S26) |
| m | 961.46985(A1244, R7996, S48) |

FIG. 4D

| | |
|---|---|
| a | 1268.62231(A1649, R6325, S24) |
| b | 1274.69116(A120275, R16367, S3684) |
| c | 1277.63147(A25844, R7535, S376) |
| d | 1284.59766(A13352, R9354, S212) |
| e | 1290.68298(A2570, R16729, S71) |

FIG. 4E

| | |
|---|---|
| a | 3159.43994(A299, R12060, S61) |
| b | 3230.55737(A296, R9956, S50) |
| c | 3280.42627(A302, R9015, S36) |
| d | 3293.57373(A6025, R11298, S1262) |

FIG. 4F

| | |
|---|---|
| a | 800.40289(A347, R8599, S21) |
| b | 839.40546(A316, R12548, S40) |
| c | 850.42584(A640, R11601, S67) |
| d | 889.42834(A371, R13799, S39) |
| e | 898.50269(A3921, R13732, S547) |
| f | 927.48894(A562, R13376, S62) |
| g | 936.42114(A867, R16096, S144) |
| h | 957.51190(A358, R8893, S31) |
| i | 958.48816(A1454, R14759, S195) |

FIG. 4G

| | |
|---|---|
| a | 1260.62341(A755, R15936, S34) |
| b | 1277.62939(A10239, R17679, S781) |
| c | 1283.71399(A21495, R16483, S1618) |
| d | 1288.67834(A540, R14581, S21) |
| e | 1311.73730(A920, R14903, S42) |

FIG. 4H

| | |
|---|---|
| a | 3159.56885(A121, R20941, S32) |
| b | 3230.65479(A511, R10493, S91) |
| c | 3291.56689(A143, R7477, S26) |
| d | 3293.67017(A4378, R11949, S955) |

1. A method for isolating N-terminal peptides from a protein or mixture of proteins, comprising:
(a) protecting the N-terminal amino acid in the protein or in proteins of the protein mixture,
(b) fragmenting the protein or the protein mixture from (a) to obtain a protein peptide mixture, and
(c) reacting the protein peptide mixture from (b) with an aminopeptidase, whereby said N-terminal peptides are isolated.

2. A method for isolating C-terminal peptides from a protein or mixture of proteins, comprising:
(a) protecting the C-terminal amino acid in the protein or in proteins of the protein mixture,
(b) fragmenting the protein or the protein mixture from (a) to obtain a protein peptide mixture, and
(c) reacting the protein peptide mixture from (b) with a carboxypeptidase,
whereby said C-terminal peptides are isolated.

3. A method for isolating, from a protein or mixture of proteins, N-terminal peptides in which the N-terminal amino acid has been blocked in vivo, comprising:
(i) fragmenting the protein or the protein mixture to obtain a protein peptide mixture, and
(ii) reacting the protein peptide mixture from (i) with an aminopeptidase,
whereby said N-terminal peptides in which the N-terminal amino acid has been blocked in vivo are isolated.

4. The method according to claim 3, wherein said N-terminal peptides in which the N-terminal amino acid has been blocked in vivo include N-terminal α-NH₂ acetylation or N-terminal formylation or pyroglutaminyl formation on N-terminus of peptides.

5. A method for isolating, from a protein or mixture of proteins, C-terminal peptides in which the C-terminal amino acid has been blocked in vivo, comprising:
(i) fragmenting the protein or the protein mixture to obtain a protein peptide mixture, and
(ii) reacting the protein peptide mixture from (i) with a carboxypeptidase,
whereby said C-terminal peptides in which the C-terminal amino acid has been blocked in vivo are isolated.

6. The method according to claim 5, wherein said C-terminal peptides in which the C-terminal amino acid has been blocked in vivo include C-terminal Asn cyclisation or C-terminal cholesterol addition.

7. The method according to any of claim 1, 2, 3 or 5, wherein said aminopeptidase or said carboxypeptidase is non-specific.

8. The method according to any of claim 1, 2, 3 or 5, which uses a combination of two or more aminopeptidases with complementary specificities, or of two or more carboxypeptidases with complementary specificities.

9. The method according to any of claims 1, 2, 3 or 5, wherein said aminopeptidase or said carboxypeptidase is naturally occurring, or is engineered to obtain optimal or evolved enzymatic characteristics for progressive removal of unprotected amino acids.

10. The method according to any of claims 1, 2, 3 or 5, further comprising separating said N-terminal or C-terminal peptides from amino acids.

11. The method according to any of claims 1, 2, 3 or 5, further comprising:
(i) separating the isolated N-terminal peptides or C-terminal peptides into fractions of peptides via a single- or multi-dimensional separation process; and
(ii) identifying one or more N-terminal peptides or C-terminal peptides from one or more of said fractions,
whereby said identified N-terminal peptides or C-terminal peptides represent one or more proteins from the mixture of proteins.

12. The method according to claim 11, whereby proteins differentially present between different samples are identified, preferably to identify biomarkers.

13. The method according to claim 11, whereby endogenous proteolytic events and cleavage sites in proteins are identified.

14. A kit specifically designed for isolating N-terminal peptides or C-terminal peptides from a protein or mixture of proteins, comprising one or more or all of the following elements:
an agent for effecting protection of the N-terminal amino acid in the protein or in proteins of the protein mixture, and/or an agent for effecting protection of the C-terminal amino acid in the protein or in proteins of the protein mixture;
an agent for effecting fragmentation of the protein or the protein mixture into a protein peptide mixture;
one or more aminopeptidases and/or one or more carboxypeptidases;
a separation means for separating peptides and amino acids, preferably a size exclusion chromatographic means, such as, more preferably, a size exclusion chromatographic column, said size exclusion chromatographic means having a separation cut-off of between about 400 Da and about 1000 Da, more preferably between about 500 Da and about 800 Da, even more preferably of about 600 Da or about 700 Da.


SEQUENCE LISTING (6 sequences)

SEQ ID NO: 1 - protein, 13 aa, Artificial Sequence
  Chemically synthesized peptide P1380
IPMYSIITPNVLR

SEQ ID NO: 2 - protein, 16 aa, Artificial Sequence
  Chemically synthesized peptide P1384
SELEEDIIPEEDIISR

SEQ ID NO: 3 - protein, 11 aa, Artificial Sequence
  Chemically synthesized peptide L2
LADGGATNQGR

SEQ ID NO: 4 - protein, 14 aa, Artificial Sequence
  Chemically synthesized peptide L3
ELSEALGQIFDSQR

SEQ ID NO: 5 - protein, 9 aa, Artificial Sequence
  Chemically synthesized peptide L4
STHTLDLSR

SEQ ID NO: 6 - protein, 12 aa, Artificial Sequence
  Chemically synthesized peptide L5
GLNLTEDTYKPR